(19) United States
(12) Patent Application Publication
(10) Pub. No.: US 2024/0398218 A1
(43) Pub. Date: Dec. 5, 2024

(54) ELECTRONIC ASSEMBLIES FOR MEDICAL DEVICES

(71) Applicant: Boston Scientific Scimed, Inc., Maple Grove, MN (US)

(72) Inventors: Eric Noble LENT, Roberts, WI (US); Eric F. BROWN, Wilmington, MA (US); James Eric BLOOD, Shoreview, MN (US); Thao N. NGUYEN, Lakeville, MN (US)

(73) Assignee: Boston Scientific Scimed, Inc., Maple Grove, MN (US)

(21) Appl. No.: 18/675,418

(22) Filed: May 28, 2024

Related U.S. Application Data
(60) Provisional application No. 63/504,813, filed on May 30, 2023.

(51) Int. Cl. A61B 1/05; A61B 1/07

(52) U.S. Cl. CPC A61B 1/05; A61B 1/07

(57) ABSTRACT

An electronic assembly for a medical device may include a circuit board. The electronic assembly may also include an imaging device mounted on a first edge of a first portion of the circuit board and a lighting element mounted on a second edge of a second portion of the circuit board.

### CROSS-REFERENCE TO RELATED APPLICATIONS

This application claims the benefit of priority under 35 U.S.C. § 119 from U.S. Provisional Application No. 63/504,813, filed May 30, 2023, which is incorporated by reference herein in its entirety.

### TECHNICAL FIELD

The disclosure relates generally to electronic assemblies for medical devices and associated methods. More specifically, aspects of the disclosure pertain to assemblies including components that are positioned at a distal tip of a medical device, such as an endoscope.

### BACKGROUND

In a medical procedure, an operator may insert a medical device, such as an endoscope or other type of scope, into a body lumen of a subject. The operator may navigate the distal tip to a location where a procedure is to be performed. The operator may pass accessory devices (e.g., instruments) through a working channel of the medical device to assist with performing certain diagnostic or therapeutic procedures. The distal tip of the medical device may include elements for providing visualization of the body lumen, such as lighting elements and/or imaging devices. The distal tip of the medical device also may include other features, such as an opening of the working channel, openings for air/water and suction, and/or an elevator. It may be desirable to increase a size of the working channel and its associated opening or introduce additional elements to the distal tip of the medical device without increasing an outer diameter of the medical device. It may further be desirable to deliver images of high quality to an operator. Therefore, a need exists for electronic assemblies for distal tips of medical devices.

### SUMMARY

Each of the aspects disclosed herein may include one or more of the features described in connection with any of the other disclosed aspects.

An electronic assembly for a medical device may include: a circuit board; an imaging device mounted on a first edge of a first portion of the circuit board; and a lighting element mounted on a second edge of a second portion of the circuit board.

Any of the aspects disclosed herein may include any of the following features, in any combination. The lighting element may be a first lighting element, the electronic assembly further comprising a second lighting element mounted on a third edge of a third portion of the circuit board. The first lighting element may be oriented approximately perpendicularly to the second lighting element. The circuit board may include an arm portion. The arm portion may include the second portion of the circuit board. At least a segment of the arm portion may be a flexible segment. The flexible segment may include at least one bend. The first edge may be a distal edge of the first portion. The first edge may be disposed between two contacts of the imaging device. The second edge may be disposed between two contacts of the lighting element. The imaging device may face approximately parallel to a plane of the first portion of the circuit board. The circuit board may include a proximally extending portion configured to extend through at least a portion of a shaft of the medical device. The proximally extending portion may extend into a handle of the medical device. The proximally extending portion may include at least one winding. The proximally extending portion may include at least one coplanar wave guide. The proximally extending portion may further include a ground plane. The proximally extending portion may include a copper plane. At least a portion of the copper plane may be cross-hatched.

In another aspect, an electronic assembly for a medical device may include: a distal portion configured to be disposed within a distal tip of the medical device; at least one component mounted on a circuit board of the distal portion; and a proximally extending portion configured to be disposed within a shaft of the medical device. The proximally extending portion may include a circuit board.

Any of the aspects disclosed herein may have any of the following features in any combination. The circuit board of the distal portion and the circuit board of the proximally extending portion may be one single, unitary structure. The proximally extending portion may include at least one winding. The at least one component may be mounted to an edge of the circuit board of the distal portion.

In another aspect, an electronic assembly of a medical device may comprise: a distal portion configured to be disposed within a distal tip of the medical device; and a proximally extending portion configured to extend through a shaft and into a handle of the medical device. A portion of the proximally extending portion within the shaft may include a circuit board.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIGS. 1A-1B depict an exemplary medical device.

FIG. 2 depicts an exemplary electronic assembly for use with a medical device, such as the medical device of FIGS. 1A-1B.

FIGS. 3A-3B depict another exemplary electronic assembly for use with a medical device, such as the medical device of FIGS. 1A-1B.

FIGS. 4A-4B depict a further exemplary electronic assembly for use with a medical device, such as the medical device of FIGS. 1A-1B.

FIGS. 5A-5B depict an additional exemplary electronic assembly for use with a medical device, such as the medical device of FIGS. 1A-1B.

FIGS. 6A-6B depict another exemplary electronic assembly for use with a medical device, such as the medical device of FIGS. 1A-1B.

### DETAILED DESCRIPTION

It may be understood that both the foregoing general description and the following detailed description are exemplary and explanatory only and are not restrictive of the invention, as claimed. As used herein, the terms “comprises,” “comprising,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The drawings may include arrows labeled “P” and “D,” indicating proximal and distal directions, respectively. The term “approximately,” or like terms (e.g., “substantially”), includes values +/−10% of a stated value.

A medical device, such as an endoscope may be inserted into a body lumen of a subject in order to perform a medical procedure. The distal tip of the medical device may include electronic assemblies including various components, such as lighting elements (e.g., light emitting diodes (LEDs), optical fibers or other light guides, or a combination thereof), imaging elements (e.g., cameras, other components having imagers, or other optical elements (e.g., lenses)), associated electronic elements (e.g., capacitors, diodes, resistors, and the like). Electronic components may include circuit boards (e.g., printed circuit boards) with various elements mounted thereon.

It may be desirable for electronic assemblies to occupy a small amount of space while retaining quality of performance and manufacturing. One exemplary manner of conserving space while retaining quality of performance and manufacturing is to include various components that are orthogonally mounted to an edge of a circuit board. The circuit board may be rigid, flexible, or a combination of both. Various arrangements of the circuit board and components mounted thereon are possible, depending upon requirements of the medical device. Additionally or alternatively, an electronic assembly may include a circuit board that extends from a distal tip of a medical device, through a shaft of the medical device, to a proximal end of a medical device (e.g., between a distal tip and a handle of the medical device). Such an assembly may include a circuit board that has a helical or other coiled shape, which extends through the shaft of the medical device. Furthermore, the assemblies may include circuit boards that have features to control impedance and/or provide electromagnetic shielding.

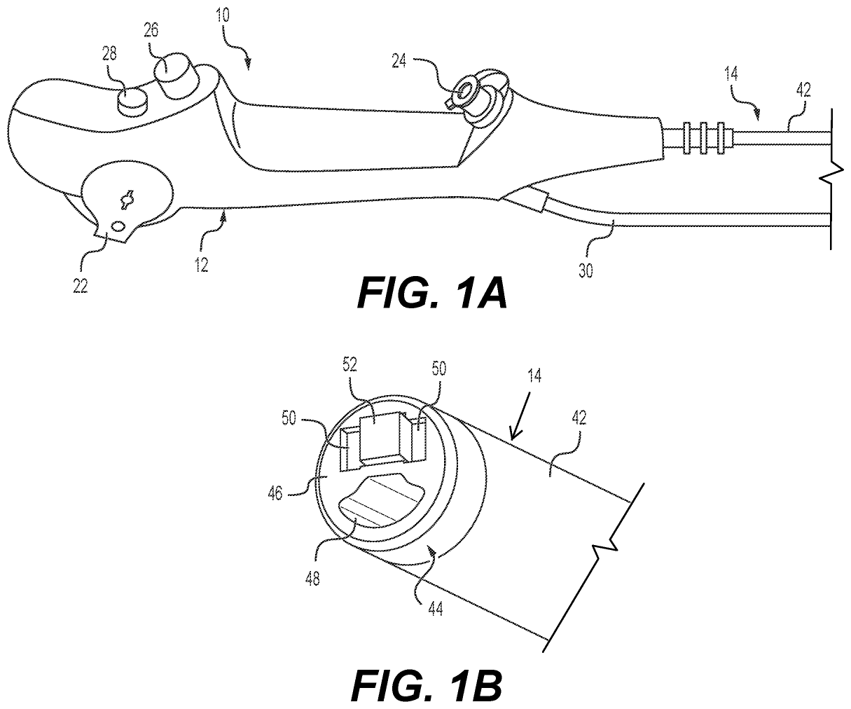

FIGS. 1A and 1B depict aspects of an exemplary medical device 10. FIG. 1A depicts a proximal portion of medical device 10. FIG. 1B depicts a distal tip 44 of medical device 10. Medical device 10 may include a handle portion 12 for gripping and operation by an operator, and an insertion portion 14 for at least partial insertion into a body (e.g., a body lumen) of a subject. As shown in FIGS. 1A and 1B, medical device 10 may include an endoscope. Although the disclosure may refer at different points to an endoscope, it will be appreciated that, unless otherwise specified, duodenoscopes, endoscopes, gastroscopes, endoscopic ultrasonography (“EUS”) scopes, colonoscopes, ureteroscopes, bronchoscopes, laparoscopes, cystoscopes, aspiration scopes, sheaths, catheters, or any other suitable delivery device or medical device may be used in connection with the elements and assemblies described herein.

Handle portion 12 may include a lever 22, for example, on a proximal portion of handle portion 12. Lever 22 may help to facilitate articulation/steering of insertion portion 14, including distal tip 44. Although lever 22 is depicted in FIG. 1A, it will be appreciated that any suitable actuator(s) may be used in addition to or in place of lever 22, such as one or more knobs, buttons, sliders, or joysticks. A port 24 of handle portion 12 (e.g., on a proximal portion of handle portion 12) may provide access to a lumen or working channel of medical device 10. An operator may insert an instrument or other device into port 24 and may extend the instrument or other device distally through the working channel. The working channel may extend longitudinally through a length of insertion portion 14. Handle portion 12 may also include a suction valve 26, for example, on a proximal portion of handle portion 12 and on an opposing side from lever 22. An operator may connect suction valve 26 to a source of suction, and may operate suction valve 26 to generate suction through insertion portion 14 (e.g., through the working channel). Handle portion 12 may additionally or alternatively include other types of valves, such as air and/or water valves, or valves that perform a combination of functions.

An image capture button 28 of handle portion 12 may enable an operator to capture a still image from an imaging device 52 (shown in FIG. 1B and described in further detail below) during a procedure. Image capture button 28 may be positioned on a proximal portion of handle portion 12, for example, adjacent suction valve 26. Additionally or alternatively, image capture button 28 may enable an operator to capture video or to perform other functions to control medical device 10. An umbilicus 30 may extend from handle portion 12 (e.g., from a distal portion of handle portion 12) and may carry wires, cables, and/or conduits for providing, for example, power, signals, or fluids to and/or from handle portion 12. For example, umbilicus 30 may connect handle portion 12 to one or more user interfaces, monitors, displays, etc.

Insertion portion 14 may include a shaft 42 extending distally from handle portion 12. Shaft 42 may have any suitable properties. For example, shaft 42 may be flexible and may have wires, tubes, or other features passing therethrough. Distal tip 44 of medical device 10, depicted in FIG. 1B, may be disposed at a distal end of shaft 42. As shown in FIG. 1B, distal tip 44 may include a distalmost face 46. Distalmost face 46 may define a working channel opening 48. The working channel may extend between port 24 and working channel opening 48, such that instruments or other devices may be passed through port 24, through the working channel, and out of working channel opening 48. An instrument extending distally of working channel opening 48 may be used to perform a medical procedure on a subject.

Distal tip 44 may also include imaging components, such as one or more lighting elements 50 and one or more imaging devices (e.g., camera) 52. Although two lighting elements 50 and one imaging device 52 are depicted in FIG. 1B, it will be appreciated that alternative numbers of lighting elements 50 and imaging device 52 may be utilized. Alternatively, lighting elements 50 and imaging device 52 may be combined into a single device. Lighting elements 50 may include LEDs or any suitable alternative light source. Imaging device 52 may be configured to take video and/or still images. Imaging device 52 may provide a signal to a monitor (not shown), so that an operator may view a visual image provided by imaging device 52 while navigating medical device 10 through a body lumen of a subject.

As depicted in FIG. 1B and described above, medical device 10 may be “forward-facing.” In other words, features of distal tip 44 (e.g., working channel opening 48, lighting elements 50, and imaging device 52) may face distally (i.e., forward of distalmost face 46. This disclosure also encompasses other configurations of distal tip 44. For example, medical device 10 may be “side-facing.” In a side-facing embodiment, working channel opening 48, lighting elements 50, and/or imaging device 52 may be disposed on a radially outer side of distal tip 44, so that they point in a radially outward direction, approximately perpendicularly to a longitudinal axis of insertion portion 14. Medical device 10 may additionally include some components that are forward-facing and other components that are side-facing.

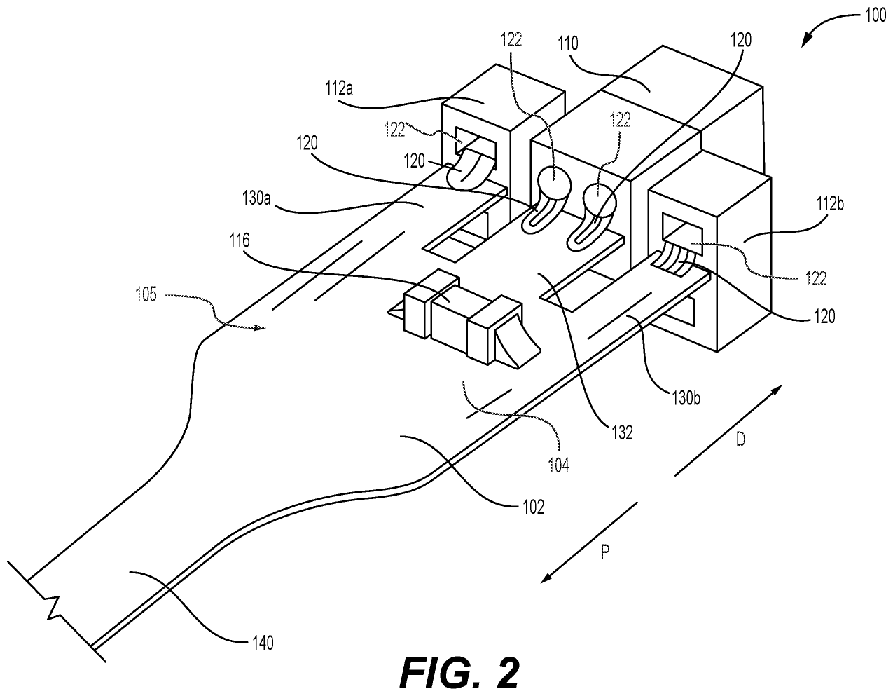

FIG. 2 depicts an exemplary electronic assembly 100, which may be disposed at least partially within distal tip 44 (FIG. 1B). Electronic assembly 100 may include a substrate 102, which may be, for example, a printed circuit board (PCB). Substrate 102 may include one or more connection points (e.g., pads or leads) for various components mounted thereon and/or for connecting wires and/or cables (not shown) that extend proximally from substrate 102, through shaft 42, to handle 12. Substrate 102 also may include traces connecting components to one another and/or to wires or cables. As shown in FIG. 2, substrate 102 may be approximately flat/planar. However, substrate 102 may have any alternative configuration (e.g., a bent and/or curved configuration). Substrate 102 may be flexible, rigid, or a combination of both.

Electronic assembly 100 may include an imaging device 110 coupled thereto. Imaging device 110 may include or be a camera, and may have any of the properties of imaging device 52. For example, imaging device 110 may include an image sensor and/or one or more lenses. Electronic assembly 100 also may include one or more lighting elements, such as two lighting elements 112a, 112b depicted in FIG. 2. Lighting elements 112a, 112b may be, for example, light emitting diodes (LEDs) and may have any of the properties of lighting elements 50.

As shown in FIG. 2, one or more of imaging device 110 or lighting elements 112a, 112b may be mounted normally/orthogonally to substrate 102. In other words, imaging device 110 and/or lighting elements 112a, 112b may face a direction that is perpendicular to/orthogonal to a normal (e.g., a normal line) of a plane of a portion of substrate 102 to which the respective element (imaging device 110 and/or lighting elements 112a, 112b) is coupled. In other words, imaging device 110 and/or lighting elements 112a, 112b may face a direction that is approximately parallel to the plane of a portion of substrate 102 to which the respective element is coupled. When it is said that imaging device 110 and/or lighting elements 112a, 112b “face” a direction, it is meant that an operative portion of the component is directed in that direction.

As shown in FIG. 2, imaging device 110 and/or lighting elements 112a, 112b may be affixed to an edge (e.g., one or more distal edges) of substrate 102. The edge may extend between surfaces of substrate 102 (e.g., between a first/front surface 104 and a second/back surface (not shown) opposite to first surface 104). In general, a given portion of substrate 102 or the other substrates described herein will have two surfaces (top and bottom), with a plurality of edges extending between the two surfaces. Although imaging device 110 and lighting elements 112a, 112b are depicted as being mounted to an edge of substrate 102, it will be appreciated that other components may be additionally mounted to an edge of substrate 102, or that one or more of imaging device 110 or lighting elements 112a, 112b may be mounted to a surface (e.g., first surface 104) of substrate 102.

Components such as imaging device 110 and/or lighting elements 112a, 112b may be coupled (e.g., conductively coupled) to substrate 102 via one or more connection(s) 120 (e.g., solder fillets). In some examples, components such as imaging device 110 and/or lighting elements 112a, 112b may include contacts 122 thereon for making connection(s) 120. For example, imaging device 110 and/or lighting elements 112a, 112b (or other components) may include solder balls (e.g., of SAC 305 solder) or solder bumps (e.g., of gold) on a side (e.g., a rear side) thereof that are used to make connection(s) 120. For example, components such as imaging device 110 and/or lighting elements 112a, 112b, may include a ball grid array (BGA). Such examples of contacts 122 are merely exemplary, and other structures may be utilized. Such contacts 122 may be connected to elements of substrate 102 (e.g., pads of substrate 102) via, e.g., hot air reflow, other soldering methods, and/or conductive epoxies to form connection(s) 120. In examples, a strength of connections 120 may be increased using epoxies or adhesives that are conductive or non-conductive. One or more components (e.g., imaging devices 110 and/or lighting elements 112a, 112b) may be overmolded and/or encapsulated along with portions (or an entirety of) substrate 102. Overmolding and/or encapsulation may provide protection from fluids, increase strength, and/or aid biocompatibility.

Pads or other connection points of substrate 102 for connecting to the components (not separately visible in FIG. 2 and integrated into connections 120) may be disposed at or near the edge to which the component is to be attached. Such pads/connection points may be disposed on one or more surfaces of substrate 102 (e.g., first surface 104 or the opposite surface). In some examples, pads/connecting points may be located at corresponding locations on opposite surfaces of substrate 102. In such examples, a normal line extending through substrate 102 may pass through two pads/connection points (one on each surface). Thus, connections 120 may be formed on both a top and a bottom side of substrate 102. The components may additionally or alternatively be fixed to substrate 102 using an adhesive, via encapsulation, etc.

Components affixed to substrate 102 may have different numbers of contacts 122, and substrate 102 may have a corresponding number of connection points/pads. For example, lighting element 112a may have two contacts 122, one on each side of substrate 102. Each face of substrate 102 (e.g., first surface 104 and the opposite surface) may include one connection point, for connecting to a corresponding contact 122 of lighting element 112a. Likewise, lighting element 112b may have two contacts 122, one on each side of substrate 102. Each face of substrate 102 (e.g., first surface 104 and the face opposite first surface 104) may include one connection point, for connecting to a corresponding contact 122 of lighting element 112b. Thus, for each lighting element 112a, 112b, two connections 120 may be formed to electrically couple contacts 122 of lighting element 112a, 112b to a connection point of substrate 102. Imaging device 110 may include four contacts 122. In the example shown in FIG. 2, imaging device 110 may have two contacts 122 on each side of substrate 102, as described below. For example, the four contacts 122 of imaging device 110 may be arranged in two rows (parallel to a plane of first surface 104) and two columns (perpendicular to a plane of first surface 104). One row of contacts 122 may be on a side of substrate 102 facing first surface 104, and the other row of contacts 122 may be on the opposite side of substrate 102. Four connections 120 may be formed between contacts 122 of imaging device 110 and connection points of substrate 102. As shown in FIG. 2, first surface 104 may include four total connections 120 formed thereon-one for lighting element 112a, two for imaging device 110, and one for lighting element 112b. A surface of substrate 102 opposite first surface 104 may have four corresponding connections 120. The arrangements described above are merely exemplary, and the components affixed to substrate 102 may have any suitable arrangement of contacts 122.

Substrate 102 and/or components affixed thereto (e.g., imaging device 110 and/or lighting elements 112a, 112b) may be configured so as to facilitate mounting of the components to an edge of substrate 102. For example, a thickness of substrate 102 (i.e., a length of an edge of substrate 102, which may be a distance between first surface 104 and a second surface opposite first surface 104) may be such that it fits between connection structures (e.g., balls or bumps) on a component, while not having too large of a distance between contacts 122 of the component (e.g., imaging device 110 and/or lighting elements 112a, 112b) and the pads/connection points of substrate 102. A thickness of substrate 102 may vary along a width of substrate 102, to accommodate components having different properties. For example, certain components may be larger and/or may have a larger distance between contacts 122, and substrate 102 may have a first thickness to accommodate that arrangement. Other components may be smaller and/or may have a smaller distance between contacts 122, and substrate 102 have a second thickness, smaller than the first thickness to accommodate that arrangement. A thickness of substrate 102 may be adjusted depending on an arrangement of contacts 122 in order to optimize manufacturability, yield, and/or reliability.

Substrate 102 may also have other components affixed thereto, either on an edge of substrate 102 (i.e., orthogonally mounted) or mounted on a surface thereof. For example, as shown in FIG. 2, substrate 102 may have one or more components 116 affixed to first surface 104. In some examples, component 116 may be a capacitor (e.g., a decoupling capacitor) or other component (e.g., a transient voltage suppressor diode) configured to work or otherwise operate with one or more components, for example, with imaging device 110. Component 116 may alternatively be a sensor such as a pressure, temperature, position, or orientation sensor. Component 116 may alternatively be any suitable type of electronic element for use with distal tip 44. Component 116 may be mounted to substrate 102 according to any suitable method, so as to conductively couple component 116 to conductors of substrate 102, to imaging device 110, to lighting elements 112a, 112b, or to other elements.

As shown in FIG. 2, a distal end of substrate 102 may have a first arm 130a and a second arm 130b extending distally from a body 105 of substrate 102. First lighting element 112a may be mounted on a distal edge of first arm 130a, and second lighting element 112b may be mounted on a distal edge of second arm 130b. Widths of arms 130a, 130b (in a direction in the plane of first surface 104 and perpendicular to a longitudinal, proximal-distal axis) may be configured to accommodate lighting elements 112a, 112b. For example, as shown in in FIG. 2, arms 130a, 130b may be slightly narrower than lighting elements 112a, 112b. Substrate 102 also may have a projection 132 between arms 130a, 130b. Projection 132 may have imaging device 110 mounted to a distal edge thereof. A width of projection 132 may be configured to accommodate imaging device 110. For example, as shown in FIG. 2, projection 132 may be slightly narrower than imaging device 110. Distal edges of arms 130a, 130b and projection 132 may be approximately aligned with one another (may extend approximately the same distance distally). As shown in FIG. 2, distal ends of lighting elements 112a, 112b may be recessed relative to (i.e., positioned proximal of) a distal end of imaging device 110. Such recessing of lighting elements 112a, 112b may be due to, for example, a shorter length of lighting elements 112a, 112b along a longitudinal axis (proximal-distal axis) as compared to a length of imaging device 110 and/or lengths of arms 130a, 130b and projection 132.

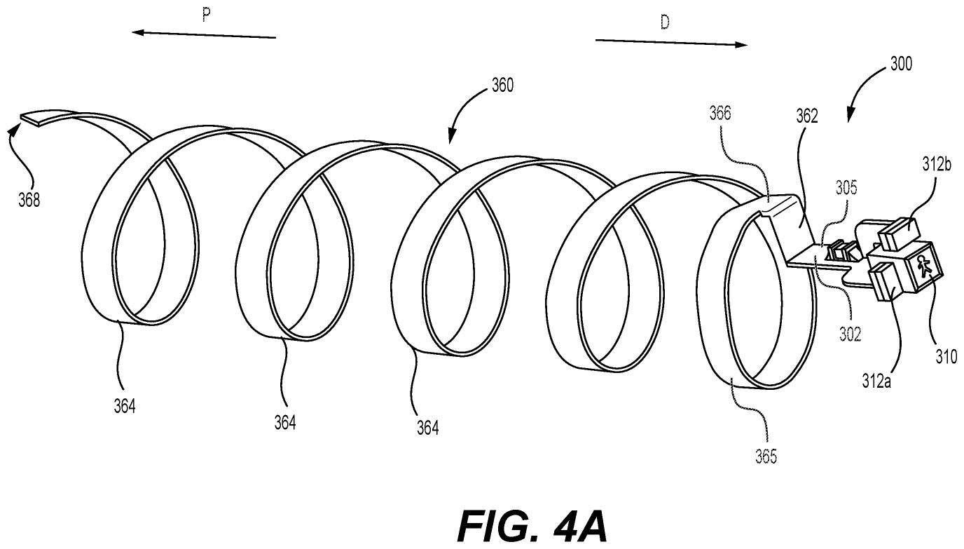

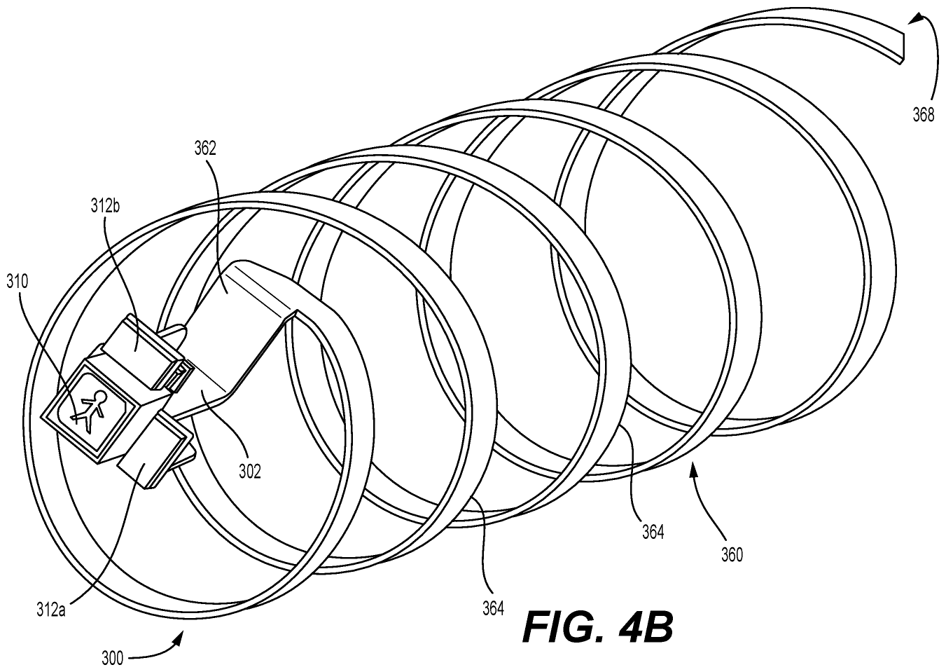

Substrate 102 also may have a proximal projection 140. Proximal projection 140 may be centered along a width of body 105 or may alternatively be arranged in any suitable manner. Proximal projection 140 may include pads or other connections (not shown) for connecting to, for example, wires or cables that extend through shaft 42. Alternatively, proximal projection may extend proximally through shaft 42, as, for example, described with respect to FIGS. 4A-4B, below.

The above configuration is merely exemplary. Any of arms 130a, 130b, projection 132, or projection 140 may be omitted. Other components may be fixed to substrate 102 in addition or in the alternative. Components described above may be omitted. For example, lighting elements 112a, 112b may be omitted, and fiber optic elements may be utilized in their place. Although substrate 102 is depicted as being flat, substrate 102 may alternatively be bent into any desired configuration.

Like any of the assemblies disclosed herein, electronic assembly 100 may be at least partially disposed in a distal tip of a medical device, such as medical device 10. For example, electronic assembly 100 may be inserted into distal tip 44 and secured therein.

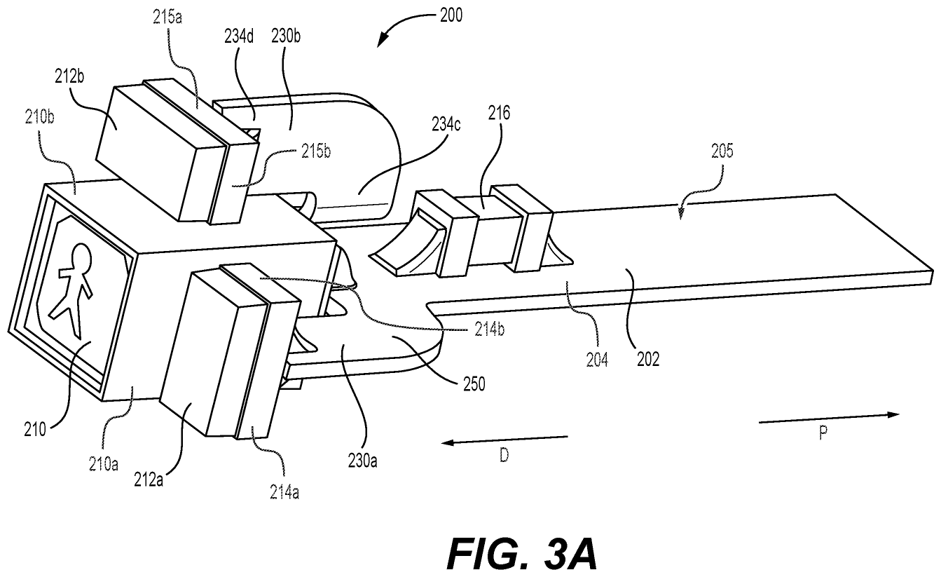

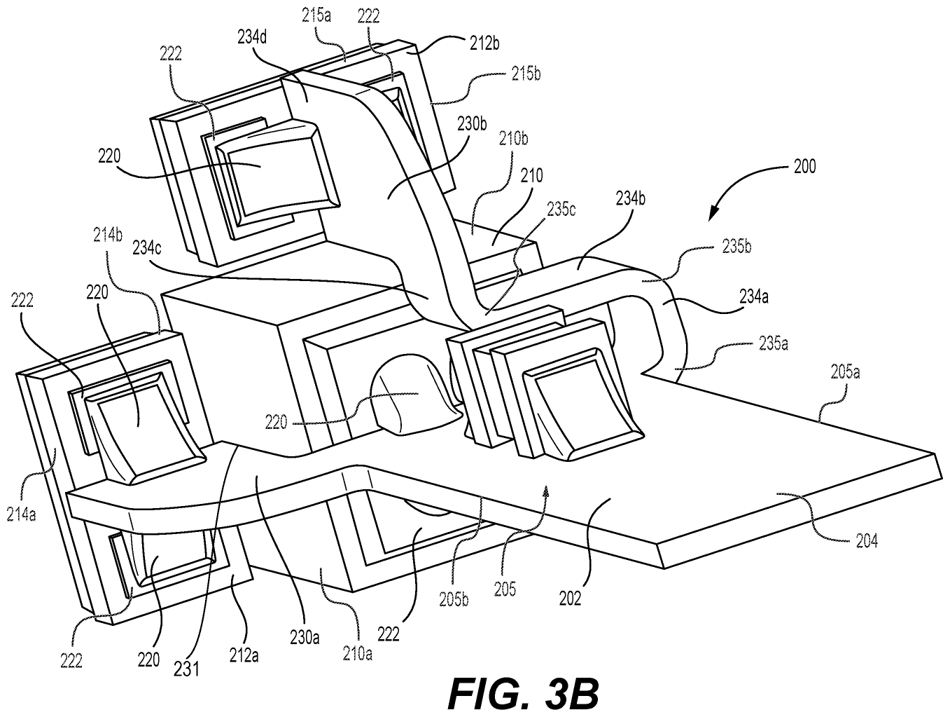

FIGS. 3A-3B depict another electronic assembly 200, which may have any of the features of electronic assembly 100, except as otherwise specified. Where feasible, similar structures are denoted by adding 100 to reference numbers associated with electronic assembly 100. Electronic assembly 200 may include a substrate 202, which may have any of the properties of substrate 102. Similar to electronic assembly 100, electronic assembly 200 may include an imaging device 210 (having any of the properties of imaging device 110) and lighting elements 212a, 212b (having any of the properties of lighting elements 112a, 112b) affixed to edges of substrate 202. Imaging device 210 may be coupled to an edge of a distal end of a body 205 (having any properties of body 105). Also similarly to electronic assembly 100, substrate 202 may include arms 230a, 230b, and lighting elements 212a, 212b may be mounted to edges of arms 230a, 230b, respectively, via connections 220, having any of the properties of connections 120. One or more components 216, having any of the properties of component(s) 116, may be coupled to body 205.

Imaging device 210 may be coupled directly to a distal end of body 205, and electronic assembly 200 may lack a structure similar to projection 132. Imaging device 210 may be centered along a width of body 205. A connection of imaging device 210 is not described in detail, as any of the aspects of connection of imaging device 110 applies to imaging device 210.

Arm 230a, like arm 130a, may extend distally from body 205 of substrate 202 and may have any of the properties of arm 130a. Arm 230a also may have a segment 250 that extends laterally outward (to a side) from body 205. An inner side 231 of arm 230a may be adjacent to imaging device 210. Arm 230a may be dimensioned so that a distal end of lighting element 212a is recessed proximally with respect to a distal end of imaging device 210.

Arm 230b may include one or more flexible portions so that arm 230b may be bent at one or more places. Although FIGS. 3A and 3B show only arm 230b as being flexible/bent, it will be appreciated that such an arrangement is merely exemplary, and any portion of substrate 202 may be flexible and/or bent. The following description of how arm 230b is bent is merely exemplary, and any other shape may be utilized for arm 230b. As shown best in FIG. 3B, arm 230b may include a first portion 234a, a second portion 234b, a third portion 234c, and a fourth portion 234d. First portion 234a may extend from a first side 205a of body 205 at a first junction 235a. As shown in FIG. 3B, first junction 235a may include a bend of approximately 90 degrees, such that a plane of first portion 234a is approximately perpendicular to a plane of body 205. In the illustrated example, first portion 234a may extend approximately along a normal direction extending from a first face 204. First junction 235a may be flexible (e.g., may be made from flexible circuit board) to accommodate such a bend. Other portions of first portion 234a may be flexible or may be rigid.

Second portion 234b may meet first portion 234a at a second junction 235b. Second junction 235b may include a bend of approximately 90 degrees, such that a plane of second portion 234b is approximately perpendicular to a plane of first portion 234a and approximately parallel to a plane of body 205. Second junction 235b may be flexible to accommodate such a bend. Other portions of first portion 234b may be flexible or may be rigid. As shown in FIG. 3B, second portion 234b may extend toward a second side 205b (opposite first side 205a) of body 205, such that a normal line to first face 204 may extend through second portion 234b.

Third portion 234c may meet second portion 234b at a third junction 235c. Third junction 235c may include a bend of approximately 90 degrees such that a plane of third portion 234c is approximately perpendicular to a plane of second portion 234b and approximately parallel to a plane of first portion 234a. As with first portion 234a, third portion 234b may extend along a normal direction extending from first face 204. Fourth portion 234d may be approximately coplanar with third portion 234c and may extend distally from third portion 234c. The arrangement of the segments of second arm 230b, described above, are merely exemplary, and any suitable arrangement may be utilized.

A width (e.g., lateral width) of second portion 234b may be approximately half of a width of body 205, such that third portion 234c extends approximately along a central longitudinal axis of body 205. Heights of first portion 234a and third portion 234c may be such that a lower edge of fourth portion 234d is slightly above an upper surface of imaging device 210. Thus, fourth portion 234d may be approximately centered over imaging device 210. In some examples, a plane of fourth portion 234d may approximately bisect imaging device 210.

As shown in FIGS. 3A and 3B, first lighting element 212a and second lighting element 212b may be mounted approximately perpendicularly to one another. In other words, contacts 222 of first lighting element 212a may be arranged vertically in FIG. 3B, while contacts 222 of second lighting element 212a may be arranged horizontally in FIG. 3B. As shown in FIGS. 3A-3B, lighting elements 212a, 212b may be approximately rectangular. Lighting element 212a may have two longer sides 214a and two shorter sides 214b. Lighting element 212b may have two longer sides 215a, and two shorter sides 215b. One of longer sides 214a of first lighting element 212a may be adjacent to a first side 210a of imaging device 210. One of longer sides 215a of second lighting element 212b may be adjacent to a second side 210b of imaging device 210. First side 210a and second side 210b may be approximately perpendicular to one another and may be adjacent to one another, for example, with ends of sides 210a and 210b being connected or otherwise abutting. Longer sides 214a, 215b may be approximately perpendicular to one another. Such an arrangement may result from arrangements of first arm 230a and fourth portion 234d of second arm 230b. A distal edge of first arm 230a may be approximately perpendicular to a distal edge of second arm 230b (i.e., a distal edge of fourth portion 234d), and a distal end of second lighting element 212b may be approximately aligned in a proximal/distal direction with a distal end of first lighting element 212a.

The arrangement of electronic assembly 200 may be useful for medical devices (e.g., medical device 10) where it is desirable to have lighting elements 212a, 212b on adjacent sides (e.g., sides 210a, 210b) of imaging device 210. Additionally or alternatively, electronic assembly 200 may facilitate positioning of various other elements of a distal end of a medical device (e.g., one or more working channels, fluidics channels, elevators, etc.). As discussed above, the particular arrangement of electronic assembly 200 is merely exemplary, and alternative arrangements/components may be utilized within the scope of the disclosure.

FIGS. 4A-4B depict another exemplary electronic assembly 300. Electronic assembly 300 may include aspects of any of electronic assemblies 100, 200. For example, electronic assembly 300 may include a substrate 302 having mounted thereon an imaging device 310 and lighting elements 312a, 312b. As shown in FIG. 4A-4B, an arrangement of substrate 302, imaging device 310, and lighting elements 312a, 312b may have an arrangement similar to assembly 200. Alternatively, the arrangement of electronic assembly 100 or any other alternative arrangement may be utilized.

Electronic assembly 300 may include a proximally extending portion 360, which may extend proximally from a body 305 (having any of the properties of bodies 105, 205) of substrate 302. When electronic assembly 300 is positioned in a medical device 10, body 305 may be at least partially within distal tip 44, and proximally extending portion 360 may extend into shaft 42. In some aspects, proximally extending portion 360 may extend through an entirety of shaft 42. For example, proximally extending portion 360 may extend from distal tip 44 to handle 12. Although proximally extending portion 360 is described as being disposed within shaft 42, it will be appreciated that proximal portions of body 305 also may be disposed within shaft 42.

In some examples, a proximal end of proximally extending portion 360 may be within or otherwise coupled to handle portion 12. In other examples, proximally extending portion 360 may extend through umbilicus 30 (e.g., toward or to capital equipment to which an end of umbilicus 30 may be connected). Proximally extending portion 360 may be or may include a circuit board that is flexible, rigid, or a combination of flexible and rigid. A proximal portion 368 of proximally extending portion 360 may include further components mounted thereon (e.g., integrated circuit(s), button(s) for controlling imaging device 310 (e.g., image capture button 28), capacitor(s), resistor(s), diode(s), or any other suitable component), and/or proximally extending portion 360 may be coupled to other elements via wires and/or cables.

As shown in FIGS. 4A-4B, proximally extending portion 360 may have a coiled/spiral/helical/solenoid shape. Proximally extending portion 360 may include a plurality of windings 364. As shown in FIGS. 4A-4B, windings 364 may be spaced apart from one another so that there are gaps between successive windings 364.

Alternatively, windings 364 may be adjacent/abutting one another. Windings 364 may be any suitable size. For example, a diameter/width of windings 364 may be such that windings 364 fit within shaft 42 (e.g., a diameter of approximately 2 mm or less or a larger, suitable, diameter). For example, windings 364 may extend within an interior of shaft 42, through a lumen defined in shaft 42. Windings 364 may be free floating within shaft 42, or may be secured to shaft 42 (e.g., to an inner wall of shaft 42). Alternatively, windings 364 may be laminated within a wall of shaft 42 or may extend externally of at least some layers of a wall of shaft 42. Windings 364 may provide flexibility to proximally extending portion 360, so that proximally extending portion may bend along with shaft 42 and may not affect a stiffness of shaft 42. In alternatives, proximally extending portion 360 may be used to modify a stiffness of shaft 42 to obtain a desired stiffness.

In alternatives, windings 364 may be omitted or may extend along only a portion of proximally extending portion 360. In aspects, spaces between windings 364 may be uniform or may be variable, or may be a combination of both. In further alternatives, proximally extending portion 360 may be twisted axially or may have alternative shapes. Proximally extending portion 360 or other portions of electronic assembly 300 may be folded/bent. For example, as shown in FIGS. 4A-4B, proximally extending portion 360 may include a segment 362 that extends between body 305 and a first (distalmost) winding 365. Segment 362 may be perpendicular to both body 305 and a distalmost portion 366 of first winding 365. Segment 362 may form a bridge between body 305 and windings 364. Electronic assembly 300 (including body 305 and proximally extending portion 360) may be made from a single, unitary piece of material (e.g., PCB). In alternatives, electronic assembly 300 may be formed from multiple pieces. For example, body 305 may be one piece, and proximally extending portion 360 may be a second piece. In alternatives, proximally extending portion 360 may be formed from multiple pieces.

Windings 364 may be formed by cutting a flexible PCB in a pattern (e.g., with curved sides of different curvature and/or length) so as to cause the flexible printed circuit board to curl into windings 364. Additionally or alternatively, windings 364 may be formed by winding PCB (e.g., a flexible PCB) around a mandrel. Windings 364 may alternatively be formed by any suitable method.

Electronic assembly 300, including proximally extending portion 360, may eliminate a need to connect wires and/or cables to substrates (e.g., substrates 102, 202, 302). Connecting such wires and/or cables (e.g., micro-coaxial cables) to pads of substrates may require specialized personnel, equipment, etc. Furthermore, assembly of such connections may be time intensive and/or may be a source of assembly errors, requiring correction or disposal. Instead, a distal portion of electronic assembly 300, including body 305, imaging device 310, and lighting elements 312a, 312b may be positioned in a distal tip 44 of an medical device 10, and proximally extending portion 360 may be passed through shaft 42, toward handle portion 12. A proximal portion of proximally extending portion 360 may be secured within handle portion 12 and/or may be passed through umbilicus 30.

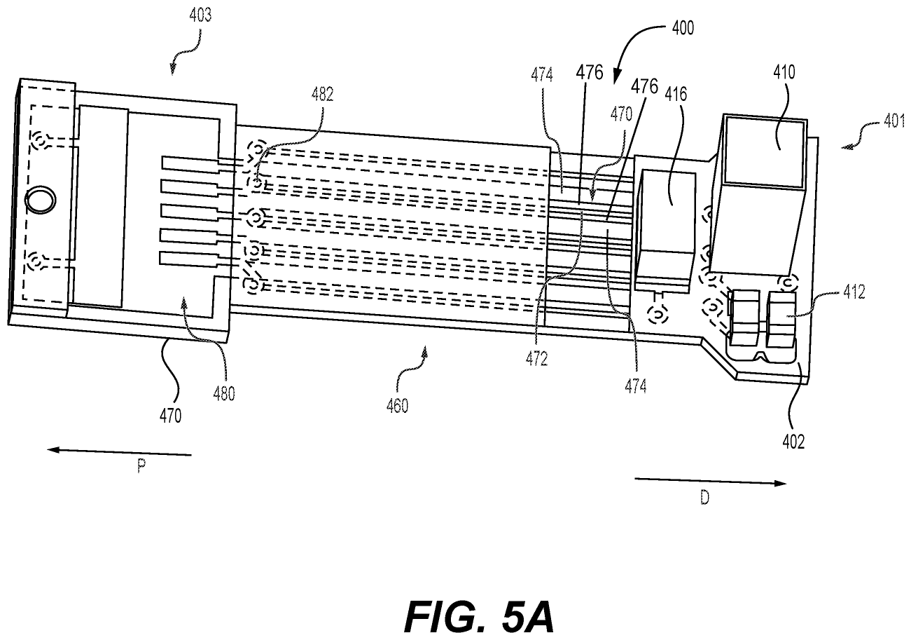

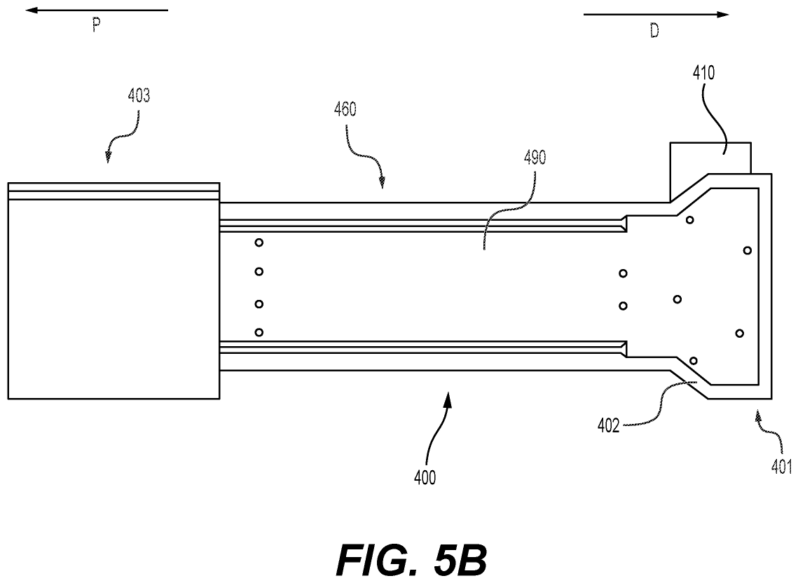

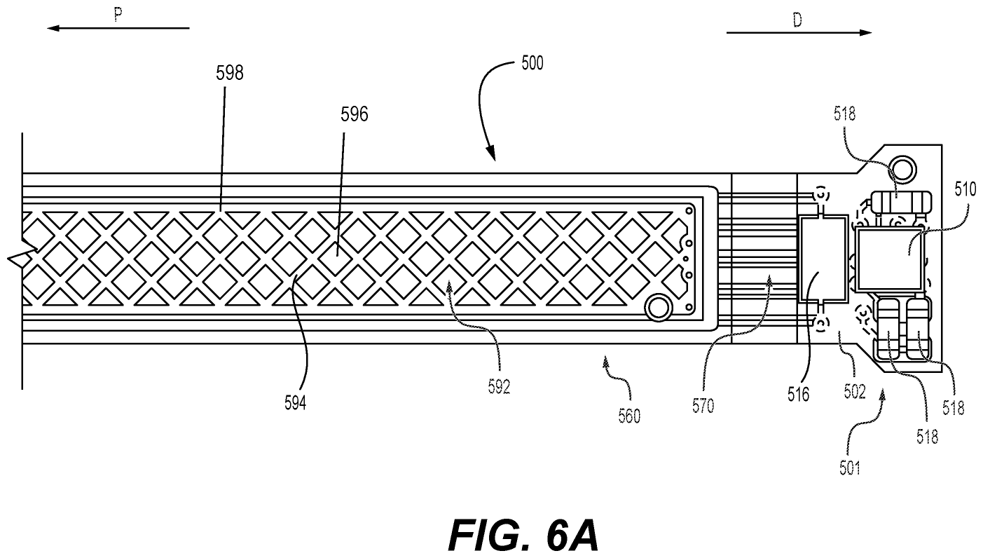

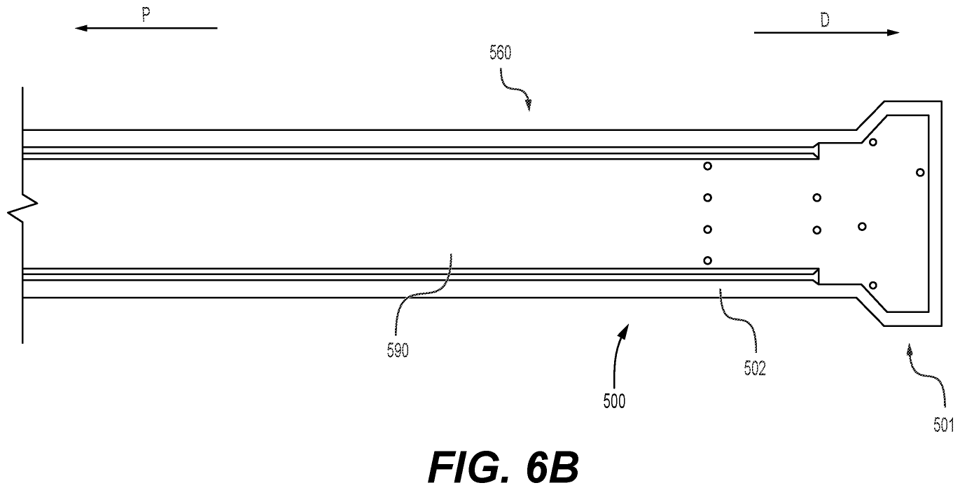

FIGS. 5A-6B depict electronic assemblies 400 and 500 that may have features that may be used in conjunction with any of the electronic assemblies described above. Although features may be described separately, it will be appreciated that any of the aspects described herein may be combined in any manner.

FIGS. 5A-5B depict electronic assembly 400, which may include one or more coplanar waveguides 470 (FIG. 5A) with an optional ground plane 490 (FIG. 5B). FIG. 5A shows a top side of electronic assembly 400, and FIG. 5B shows a bottom side of electronic assembly 400. Electronic assembly 400 may include a wave guide or embedded coplanar strip line to achieve a desired characteristic impedance and/or shielding. A strip line of electronic assembly 400 may be embedded or non-embedded. The exemplary wave guides/strip lines described herein are merely exemplary, and any suitable features may be utilized. Electronic assembly 400 may further include any of the components described above (e.g., imaging device(s), lighting element(s), capacitor(s), diode(s), resistor(s), capacitor(s), etc.)

As shown in FIG. 5A, electronic assembly 400 may include a substrate 402, having any of the features of substrates 102, 202, 302. A distal portion 401 of substrate 402 may be configured to be disposed at or near distal tip 44 of medical device 10. A distal portion of substrate 402 may have mounted thereon an imaging device 410, having any of the features of imaging devices 110, 210, 310. Although imaging device 410 is depicted as being mounted on a surface of substrate 402, instead of on an edge of substrate 402, it will be appreciated that imaging device 410 may instead be orthogonally mounted to an edge of substrate, as described above and/or depicted with respect to electronic assemblies 100, 200, and 300. Electronic assembly 400 may further include one or more lighting elements 412 mounted thereon. Lighting element(s) 412 may have any of the properties of lighting elements 112a, 112b, 212a, 212b, and 312a, 312b. Electronic assembly 400 also may include a component 416 mounted on substrate 402. Component 416 may be a resistor (e.g., a termination resistor) or any other type of component (e.g., a capacitor, such as a decoupling capacitor), such as those listed above.

Electronic assembly 400 may further include a proximally extending portion 460, having any of the properties of proximally extending portion 360. Although proximally extending portion 460 is depicted as flat, proximally extending portion 460 may include any of the configurations of proximally extending portion 360 (e.g., windings). Proximally extending portion 460 may be rigid, flexible, or a combination of both. As explained with respect to proximally extending portion 360, proximally extending portion 460 may be configured to extend through shaft 42 of medical device. 10.

Proximally extending portion 460 may include one or more coplanar wave guides 470. Coplanar wave guides 470 may include any of the features of coplanar wave guides known in the art and may provide, among other things, impedance matching. For example, each coplanar wave guide 470 may include a conducting track 472 printed onto a dielectric substrate, with a pair of return conductors 474 on either side of conducting track 472. Conducting track 472 and return conductors 474 each may be formed on a same side of substrate 402. Return conductors 474 may be separated by conducting track 472 by a gap 476 of uniform width along a length of gap 476. Dimensions of coplanar wave guides 470 may be chosen so as to provide desired properties (e.g., impedance(s)). Although FIG. 5A depicts three coplanar wave guides 470, that number is merely exemplary. Proximally extending portion 460 also may include additional conductors, such as grounds or returns to provide additional shielding to other traces/wave guides 470. Such additional conductors may be, for example, outer sides of proximally extending portion 460.

Distal ends of conducting track(s) 472, or other types of conductors (e.g., microstrips) may be electrically connected to one or more electronic components of distal portion 401 (e.g., imaging device 410, lighting elements 412, and component 416). A proximal portion 403 of electronic assembly 400 may include circuitry 480. For example, circuitry 480 may include an integrated circuit (chip) or other suitable elements. Circuitry 480 may include leads 482 for connecting to conducting track(s) 472 or other types of conductors of substrate 402. Circuitry 480 may perform any desired function. In some examples, circuitry 480 may be disposed within handle portion 12, or at a distal or proximal end of umbilicus 30 (e.g., in a connector of umbilicus 30). Alternatively, circuitry 480 may be disposed within shaft 42. In a further example, circuitry 480 may be disposed at distal portion 401.

Electronic assembly 400 also may include a ground plane 490, as shown in FIG. 5B, so that electronic assembly utilizes a conductor-backed coplanar waveguide (CBCPW). Ground plane 490 may include a conducting body. The conducting body may act as an arbitrary node of potential voltage or a common return for electric current. It may be a point of zero reference or zero volts. As shown in FIG. 5B, ground plane 490 may be on an opposite (back) side of substrate 402 from coplanar wave guide(s) 470. Ground plane 490 may have any of the features of ground planes known in the art and may interact with coplanar wave guide(s) 470 in any known way.

FIGS. 6A and 6B show a further electronic assembly 500. FIG. 6A shows a top side of electronic assembly 500, and FIG. 6B shows a bottom side of electronic assembly 500. Electronic assembly 500 may have any of the features of any of the electronic assemblies described above, and elements of electronic assembly 500 that are similar to those provided above are not described below.

Electronic assembly 500 may include a substrate 502 (having any property of substrates 102, 202, 302, 402) having a distal portion 501. Distal portion 501 may include various components mounted thereon (including any of the components of electronic assemblies 100, 200, 300, 400). Although components of electronic assembly 500 are depicted as being mounted on a face of substrate 502, it will be appreciated that any component may alternatively be mounted on an edge of substrate 502, as described with respect to electronic assemblies 100, 200, 300. Distal portion 501 may include an imaging device 510 (having any properties of imaging devices 110, 210, 310, 410), a component 516 (having any of the properties of components 116, 216, 416), and components 518 (having any of the properties of any of the components described herein). Component 516 may be, for example, a capacitor, and components 518 may be, for example, resistors.

As shown in FIG. 6A, substrate 502 may include coplanar wave guides 570, having any of the features described above, with respect to electronic assembly 400. As shown in FIG. 6B, substrate 502 may include a ground plane 590, having any of the features of ground plane 490. A top of electronic assembly 500 may further include a top plane 592. Top plane 592 may provide shielding to help mitigate or reduce electromagnetic interference. Top plane 592, additionally or alternatively, may provide for impedance matching of certain signals to provide improved image quality.

As shown in FIG. 6A, top plane 592 may be a copper plane and may include a lattice pattern 594 (e.g., of copper or any other suitable material/conductor). Alternatively, top plane 592 may include another pattern (e.g., another type of cross-hatched pattern). The pattern of top plane 592 may include regular openings 596 in lattice pattern 594 (e.g., rectangular or diamond shaped openings). The lines of lattice pattern 594 may be connected to one another (e.g., by a border 598). Top plane 592 may provide for controlled impedance while retaining flexibility (e.g., where proximally extending portion 560 is flexible, similar to examples of proximally extending portion 360). Although ground plane 590 is depicted as being solid (not hatched with a lattice pattern), it will be appreciated that ground plane 590 may additionally or alternatively include such a pattern (as may ground plane 490). Although top plane 592 and ground plane 590 are depicted as being used in conjunction with coplanar wave guides 570, it will be appreciated that coplanar wave guides 570 may be omitted. In some examples, top plane 592 and ground plane 590 may sandwich conductors carrying signals to/from distal portion 501 and may thus provide shielding benefits.

Aspects of electronic assemblies 400, 500 (e.g., coplanar wave guides 470, 570; ground planes 490, 590; and/or top plane 592) may alone, or in combination, provide for impedance matching and/or shielding. Such features may improve signals from, e.g., imaging devices 410, 510. Furthermore, such features may reduce electromagnetic waves output from a medical device 10 and limit interference from outside energy sources.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

1. An electronic assembly for a medical device, the assembly including:
a circuit board;
an imaging device mounted on a first edge of a first portion of the circuit board; and
a lighting element mounted on a second edge of a second portion of the circuit board.

2. The electronic assembly of claim 1, wherein the lighting element is a first lighting element, the electronic assembly further comprising a second lighting element mounted on a third edge of a third portion of the circuit board.

3. The electronic assembly of claim 2, wherein the first lighting element is oriented approximately perpendicularly to the second lighting element.

4. The electronic assembly of claim 1, wherein the circuit board includes an arm portion, and wherein the arm portion includes the second portion of the circuit board.

5. The electronic assembly of claim 4, wherein at least a segment of the arm portion is a flexible segment, and wherein the flexible segment includes at least one bend.

6. The electronic assembly of claim 1, wherein the first edge is a distal edge of the first portion.

7. The electronic assembly of claim 1, wherein the first edge is disposed between two contacts of the imaging device, and wherein the second edge is disposed between two contacts of the lighting element.

8. The electronic assembly of claim 1, wherein the imaging device faces approximately parallel to a plane of the first portion of the circuit board.

9. The electronic assembly of claim 1, wherein the circuit board includes a proximally extending portion configured to extend through at least a portion of a shaft of the medical device.

10. The electronic assembly of claim 9, wherein the proximally extending portion extends into a handle of the medical device.

11. The electronic assembly of claim 9, wherein the proximally extending portion includes at least one winding.

12. The electronic assembly of claim 9, wherein the proximally extending portion includes at least one coplanar wave guide.

13. The electronic assembly of claim 12, wherein the proximally extending portion further includes a ground plane.

14. The electronic assembly of claim 9, wherein the proximally extending portion includes a copper plane.

15. The electronic assembly of claim 14, wherein at least a portion of the copper plane is cross-hatched.

16. An electronic assembly for a medical device, including:
a distal portion configured to be disposed within a distal tip of the medical device;
at least one component mounted on a circuit board of the distal portion; and
a proximally extending portion configured to be disposed within a shaft of the medical device, wherein the proximally extending portion includes a circuit board.

17. The electronic assembly of claim 16, wherein the circuit board of the distal portion and the circuit board of the proximally extending portion are one single, unitary structure.

18. The electronic assembly of claim 16, wherein the proximally extending portion includes at least one winding.

19. The electronic assembly of claim 16, wherein the at least one component is mounted to an edge of the circuit board of the distal portion.

20. An electronic assembly of a medical device, comprising:
a distal portion configured to be disposed within a distal tip of the medical device; and
a proximally extending portion configured to extend through a shaft and into a handle of the medical device, wherein a portion of the proximally extending portion within the shaft includes a circuit board.
